(19) United States
(12) Patent Application Publication
(10) Pub. No.: US 2017/0361049 A1
(43) Pub. Date: Dec. 21, 2017

(54) RIGID CONTOURED RIBBON AND VARIABLE SPRING SEALING

(71) Applicant: KONINKLIJKE PHILIPS N.V., EINDHOVEN (NL)

(72) Inventors: Lauren Patricia CHODKOWSKI, PITTSBURGH, PA (US); Richard Thomas HAIBACH, VERONA, PA (US); Jonathan Sayer GRASHOW, CHESWICK, PA (US)

(73) Assignee: Koninklijke Philips N.V., Eindhoven (NL)

(21) Appl. No.: 15/539,402

(22) PCT Filed: Dec. 11, 2015
(86) PCT No.: PCT/IB2015/059530

§ 371 Date: Jun. 23, 2017

Related U.S. Application Data
(60) Provisional application No. 62/097,829, filed on Dec. 30, 2014.

(51) Int. Cl. A61M 16/06

(52) U.S. Cl. CPC A61M 16/0622

(57) ABSTRACT

A cushion support assembly for a respiratory interface device cushion is provided. The cushion support assembly includes a cushion support assembly ribbon-like body with an inner side, an outer side and a wide facial side. The cushion support assembly body facial side includes a support surface, the support surface is structured to support the respiratory interface device cushion engagement portion in a generally planar configuration. The cushion support assembly body facial side support surface is generally planar relative to the user's respiratory interface device contour radial aspect.

### CROSS-REFERENCE TO RELATED APPLICATIONS

This patent application claims the priority benefit under 35 U.S.C. §119(e) of U.S. Provisional Application No. 62/097,829, filed on Dec. 30, 2014, the contents of which are herein incorporated by reference.

### BACKGROUND OF THE INVENTION

### 1. Field of the Invention

The present invention pertains to patient interface devices for delivering a flow of breathing gas to a patient during, for example, respiratory therapy, and, in particular, to a patient interface device including a support assembly with a ribbon-like body.

### 2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), or congestive heart failure.

Non-invasive ventilation and pressure support therapies involve the placement of a patient interface device including a mask component on the face of a patient. The mask component may be, without limitation, a nasal mask that covers the patient's nose, a nasal cushion having nasal prongs that are received within the patient's nares, a nasal/oral mask that covers the nose and mouth, or a full face mask that covers the patient's face. The patient interface device interfaces the ventilator or pressure support device with the airway of the patient so that a flow of breathing gas can be delivered from a pressure/flow generating device to the airway of the patient. It is known to maintain such devices on the face of a wearer by a headgear having one or more straps adapted to fit over/around the patient's head. Because such patient interface devices are typically worn for an extended period of time, it is important for the headgear to maintain the mask component of the device in a tight enough seal against the patient's face without discomfort.

A typical cushion includes a thin sealing membrane backed by a thicker support structure, both mounted to a rigid faceplate. The face-contacting portions of the sealing membrane and support have arcuate cross-sections which are swept along the perimeter of the cushion such that the apex of the swept cross-section is contoured to the face in a longitudinal aspect. The radial contour of the face-contacting portion is not contoured to the user's face; that is, the radial contour of the face-contacting portion maintains the arcuate shape of the respective cross-section. As used herein, the type of seal provided by a cushion with such an arcuate cross-section is a “generally continuous seal.” That is, even when a sealing membrane and a support having an arcuate cross-section is biased against a user's face and deformed, the type of seal against the user's face is a “generally continuous seal.”

### SUMMARY OF THE INVENTION

One embodiment of the presently disclosed concept provides a cushion support assembly for a respiratory interface device cushion including a cushion support assembly ribbon-like body. Cushion support assembly ribbon-like body includes an inner side, an outer side and a wide facial side. Cushion support assembly body facial side includes a support surface, the support surface is structured to support the respiratory interface device cushion engagement portion in a generally planar configuration. Cushion support assembly body facial side support surface is generally planar relative to the user's respiratory interface device contour radial aspect.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

It is a further object of this invention to provide a method of using a respiratory interface device structured to engage a user's face, the user's face including a respiratory interface device contour, wherein the user's respiratory interface device contour is a shaped loop including an axial aspect and a radial aspect, the method including coupling the respiratory interface device cushion body to the cushion support assembly, positioning the respiratory interface device cushion body engagement portion to extend generally parallel to the adjacent cushion support assembly body facial side support surface, positioning the respiratory interface device over a user's respiratory orifice with the respiratory interface device cushion body engagement portion engaging the user's respiratory interface device contour.

### BRIEF DESCRIPTION OF THE DRAWINGS

FIG. 1 is a schematic view of a respiratory interface device contour for a nasal and oral respiratory interface device. FIG. 1B is a schematic view of a respiratory interface device contour for a cup-like nasal respiratory interface device. FIG. 1C is a schematic view of a respiratory interface device contour for a nasal respiratory interface device in a cradle configuration. FIG. 1D is a schematic view of a respiratory interface device contour for a nasal respiratory interface device in a sub-nasal surface sealing pillow configuration;

FIG. 2 is a schematic view of a ribbon-like body;

FIG. 3 is an isometric view of a nasal and oral respiratory interface device;

FIG. 4 is an exploded isometric view of a nasal and oral respiratory interface device;

FIG. 5 is a partial cross-sectional view of a respiratory interface device;

FIG. 5 is a back side view of a faceplate with a cushion support assembly body;

FIG. 6 is a cross-sectional view of a portion of a nasal and oral respiratory interface device. FIG. 6A is a cross-sectional view of another embodiment of a portion of a nasal and oral respiratory interface device. FIG. 6B is a cross-sectional view of another embodiment of a portion of a nasal and oral respiratory interface device. FIG. 6C is a cross-sectional view of another embodiment of a portion of a nasal and oral respiratory interface device. FIG. 6D is a cross-sectional view of another embodiment of a portion of a nasal and oral respiratory interface device;

FIG. 7 is a cross-sectional side view of another embodiment of a nasal and oral respiratory interface device;

FIG. 8 is a back side view of a faceplate with a spring element;

FIG. 9 is a detail cross-sectional view of a faceplate with a cushion support assembly body;

FIG. 10 is a detail cross-sectional view of another embodiment of a faceplate with a cushion support assembly body;

FIG. 11 is a detail cross-sectional view of another embodiment of a faceplate with a cushion support assembly body;

FIG. 12 is a detail cross-sectional view of another embodiment of a faceplate with a cushion support assembly body;

FIG. 13 is a detail cross-sectional view of another embodiment of a faceplate with a cushion support assembly body;

FIG. 14 is a detail cross-sectional view of a faceplate with a cushion support assembly body and spring element;

FIG. 15 is a detail cross-sectional view of another embodiment of a faceplate with a cushion support assembly body and spring element;

FIG. 16 is a detail cross-sectional view of another embodiment of a faceplate with a cushion support assembly body and spring element;

FIG. 17 is an isometric view of another embodiment; a nasal embodiment;

FIG. 18 is a side cross-sectional view of the embodiment shown in FIG. 17;

FIG. 19 is an top view of another embodiment; a nasal embodiment;

FIG. 20 is a side cross-sectional view of the embodiment shown in FIG. 19;

FIG. 21 is an top view of another embodiment; a nasal embodiment;

FIG. 22 is a side cross-sectional view of the embodiment shown in FIG. 21;

FIG. 23 is a flow chart of the disclosed method.

### DETAILED DESCRIPTION OF EXEMPLARY EMBODIMENTS

As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the statement that two or more parts or components “engage” one another shall means that the parts exert a force against one another either directly or through one or more intermediate parts or components. As used herein, the word “unitary” means a component is created as a single piece or unit. That is, a component that includes pieces that are created separately and then coupled together as a unit is not a “unitary” component or body. As used herein, the term “number” shall mean one or an integer greater than one (i.e., a plurality).

As used herein, a “coupling” is one element of a coupling assembly. That is, a coupling assembly includes at least two components, or coupling components, that are structured to be coupled together. It is understood that the elements of a coupling assembly are compatible with each other. For example, in a coupling assembly, if one coupling element is a snap socket, the other coupling element is a snap plug.

As used herein, “correspond” indicates that two structural components are sized and shaped to be similar to each other and may be coupled with a minimum amount of friction. Thus, an opening which “corresponds” to a member is sized slightly larger than the member so that the member may pass through the opening with a minimum amount of friction. This definition is modified if the two components are said to fit “snugly” together. In that situation, the difference between the size of the components is even smaller whereby the amount of friction increases. If the element defining the opening and/or the component inserted into the opening are made from a deformable or compressible material, the opening may even be slightly smaller than the component being inserted into the opening. With regard to surfaces, shapes, and lines, two, or more, “corresponding” surfaces, shapes, or lines have generally the same size, shape, and contours.

As used herein, “a generally continuous seal” may have a gap or may gap when the user moves. As used herein, “a more complete seal” has a gap that is shorter in length than a gap of a generally continuous seal, or, is resistant to gapping when the user moves.

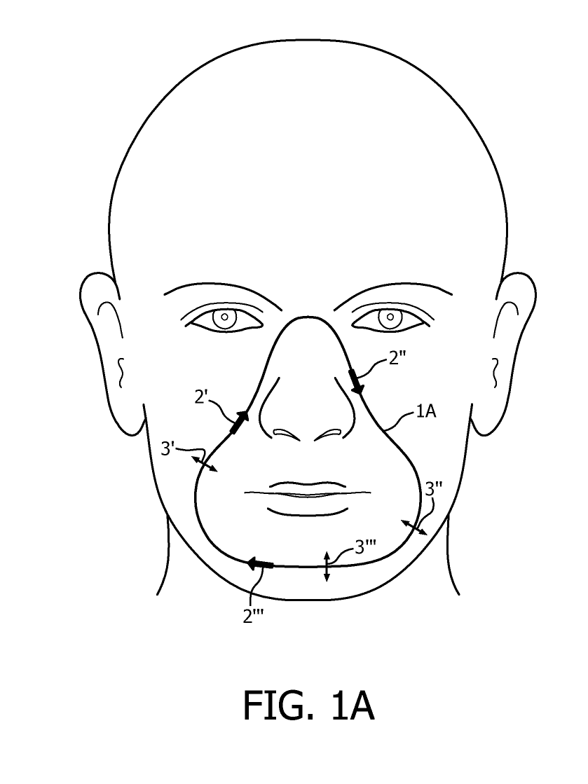

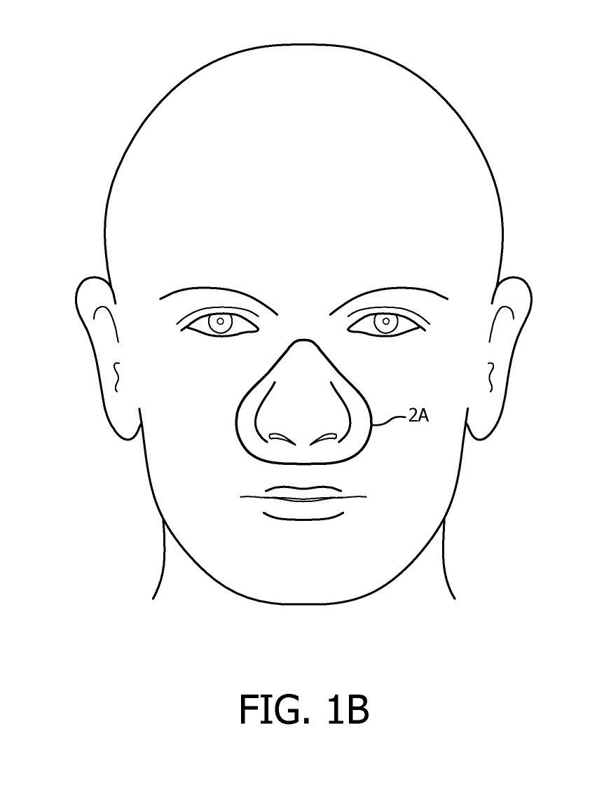

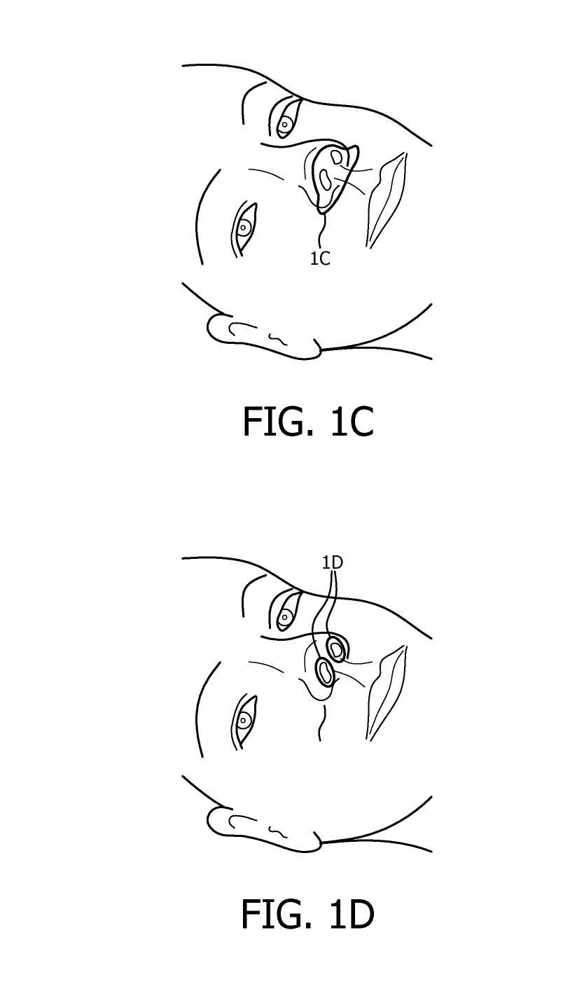

As used herein, a “respiratory interface device contour” is the perimeter of the area on a user's face that a respiratory interface device. For a nasal and oral respiratory interface device, the “respiratory interface device contour” is a loop 1A that extends around the user's nose and mouth, as shown in FIG. 1A. For a cup-like nasal respiratory interface device, the “respiratory interface device contour” is a loop 1B that extends around the user's nose and mouth, as shown in FIG. 1B. For a nasal respiratory interface device in a “cradle” configuration, the “respiratory interface device contour” is a loop 1C that extends around the lower portion of the user's nose, as shown in FIG. 1C. For a nasal respiratory interface device in a sub-nasal surface sealing pillow configuration, the “respiratory interface device contour” is a pair of loops 1D that extend around a portion of the user's upper lip and the user's nostrils, as shown in FIG. 1D.

As used herein, a “longitudinal axis” is not required to be a generally straight line. That is, a “longitudinal axis” as used herein is generally a centerline of a body which can includes curves.

As used herein, the “axial aspect” of the “respiratory interface device contour” means in the direction along the line defining the “respiratory interface device contour,” as shown by arrows 2′, 2″, 2′″ in FIG. 1A. As used herein, the “radial aspect” of the “respiratory interface device contour” means in the direction generally perpendicular to the line defining the “respiratory interface device contour” and parallel to the surface of the user's face, as shown by arrows 3′, 3″, 3′″ in FIG. 1A. Thus, a plane defined by the “axial aspect” and the “radial aspect” of the “respiratory interface device contour” means a plane at the surface of the user's face and parallel to the surface of the user's face.

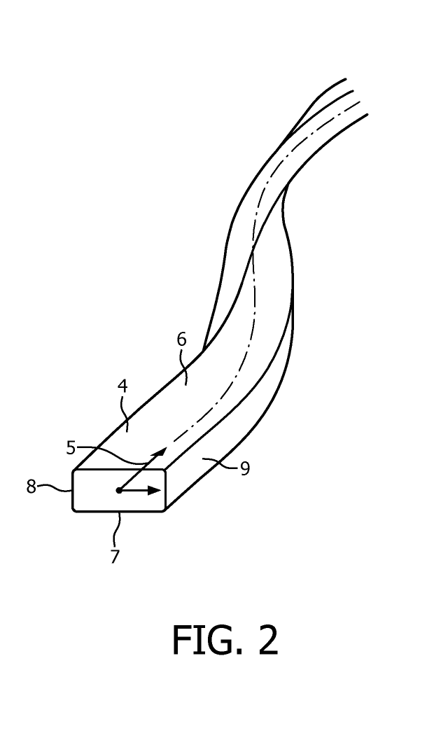

As used herein, a “ribbon-like” body, shown in FIG. 2, includes a thin, elongated body 4 having at least one surface 6 that is offset and generally parallel to the body longitudinal axis 5. The body longitudinal axis 5 may curve in three dimensions. Thus, for example, a common fabric ribbon has a generally rectangular cross-sectional shape with four surfaces that are offset and generally parallel to the longitudinal axis of the body. As shown in FIG. 2, a “ribbon-like” body with a generally rectangular cross-sectional area includes a first wide side 6, a second wide side 7, a first thin side 8, and a second thin side 9. The first and second thin sides may also be identified as “lateral sides.”

As used herein, a “ribbon seal” is a sealing surface extending in the radial aspect direction relative to a longitudinal axis. That is, a “ribbon seal” is a broad sealing surface that extends parallel to a “ribbon-like” body. Further, as used herein, a respiratory interface device that does not have a support construct with a “ribbon-like” body cannot create a “ribbon seal.” As used herein, a “ribbon seal” provides “a more complete seal” compared to a cushion with an arcuate cross-section.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

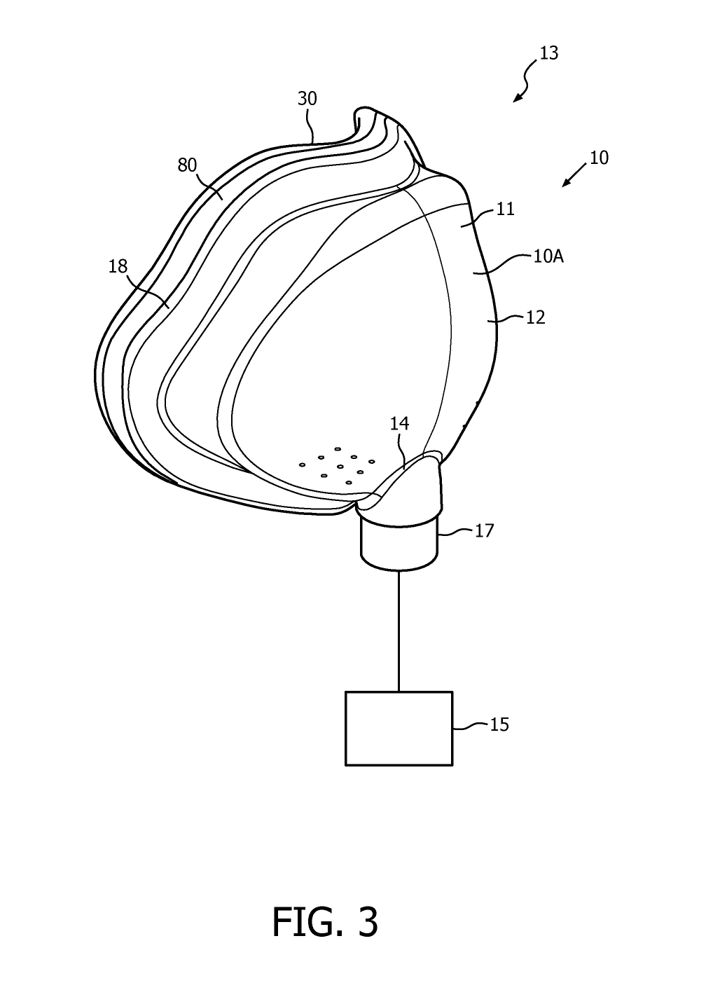

FIG. 3 shows a respiratory interface assembly 8 according to an embodiment of the invention. Respiratory interface assembly 8 includes a respiratory interface device 10 and a support assembly such as, but not limited to straps (not shown). Respiratory interface device 10 is coupled to a pressure generating system 15 (shown schematically) via a patient circuit, as is conventionally known in the art. For purposes of the present invention, the pressure generating system is any device capable of generating a flow of breathing gas or providing gas at an elevated pressure. Examples of such pressure generating systems include a ventilator, CPAP device, or variable pressure device, e.g. an auto-titrating device, proportional assist ventilation (PAV®) device, proportional positive airway pressure (PPAP) device, C-Flex™ device, Bi-Flex® device, or a BiPAP® device manufactured and distributed by Philips Respironics of Murrysville, Pa., in which the pressure provided to the patient varies with the patient's respiratory cycle so that a higher pressure is delivered during inspiration than during expiration, or other pressure support device.

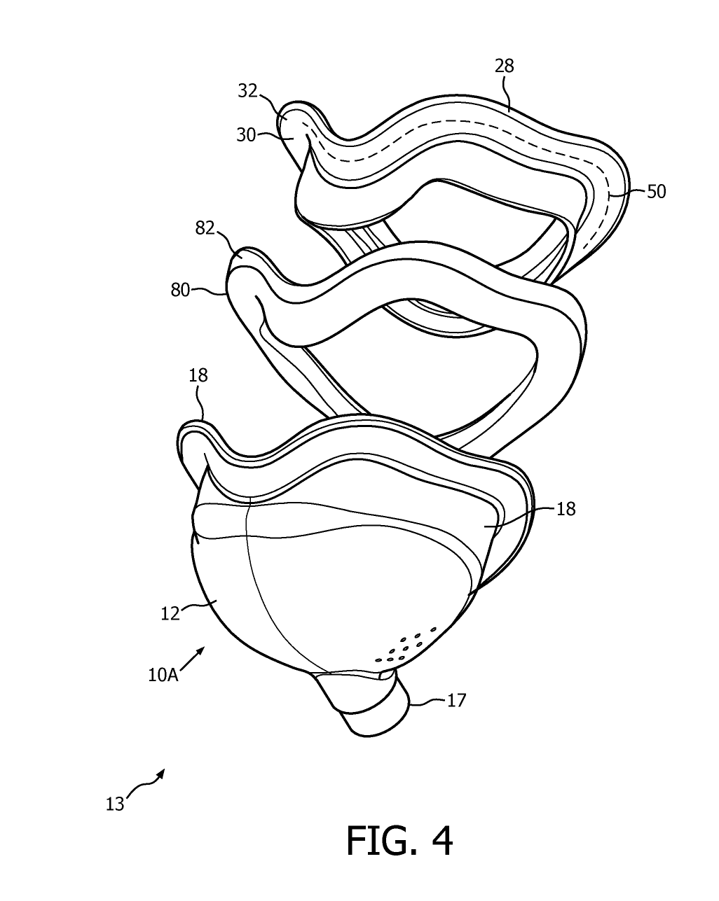

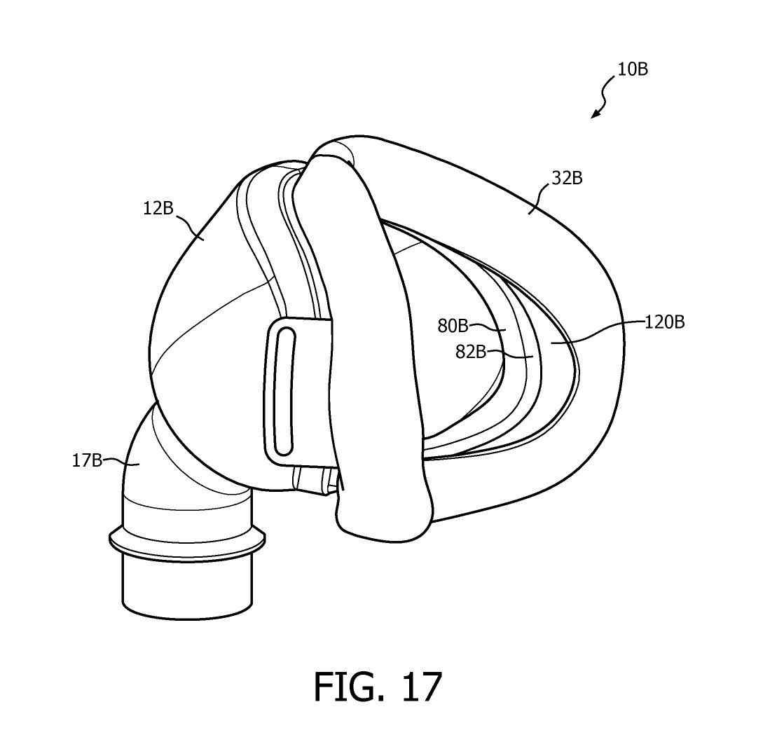

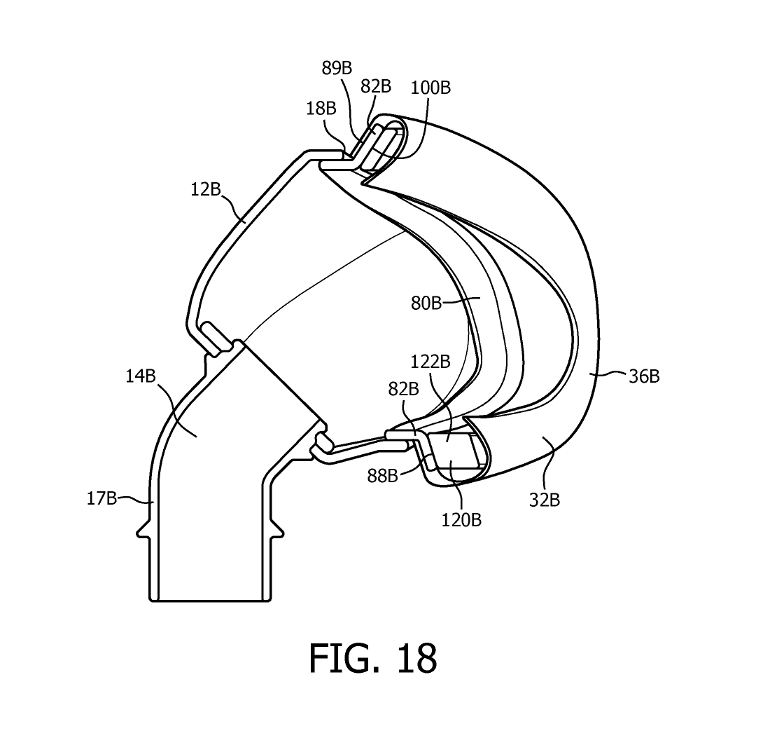

In an exemplary embodiment, shown in FIGS. 3-16, the respiratory interface device 10 is a nasal and oral respiratory interface device 10A that is structured to be disposed over a user's nose and mouth. In another exemplary embodiment, shown in FIGS. 17-18, respiratory interface device 10 is a nasal respiratory interface device 10B that is structured to be disposed over a user's nose. It is understood, however, that respiratory interface device 10 can include, without limitation, a nasal mask, nasal pillows, or any other device that provides a suitable gas flow communicating function. Thus, as used herein, the term “respiratory interface device” shall refer to any of such devices.

A nasal and oral respiratory interface device 10A includes a body 11 with a faceplate 12, a cushion 30, a cushion support assembly 80. As is known, respiratory interface device 10A is structured to be coupled to a support assembly (not shown) such as, but not limited to a number of straps. Further, and as is known, respiratory interface device 10A is structured to be coupled to, and in fluid communication with, pressure generating system 15 via a patient circuit such as, but not limited to, a number of hoses. In an exemplary embodiment, respiratory interface device body faceplate 12 (hereinafter “faceplate” 12) is a substantially rigid body. In an exemplary embodiment, shown in FIG. 3, faceplate 12 is a single piece structured to cover the user's nose and mouth. That is, respiratory interface device 10A has a peripheral contour that is, in this embodiment, structured to extend over a user's nose and mouth. In this embodiment, respiratory interface device body 11 is coextensive with faceplate 12. Faceplate 12 defines lower opening 14. Lower opening 14 can function as a gas inlet.

Gas inlet (lower opening 14) can be coupled to a coupling device 17, such as, but not limited to, a swivel conduit, for carrying gas such as air between respiratory interface device 10A and an external gas source (not shown), such as a blower, or any other suitable device. It is contemplated that the external gas source can encompass, without limitation, any gas delivery or gas generation system capable of supplying gas for consumption by a user. Non-limiting examples of various gas delivery therapies can include but are not limited to continuous positive airway pressure (CPAP) therapy, auto-titration positive airway pressure therapy, and bi-level positive airway pressure (BiPAP) therapy, as noted above. The coupling device may be any of a variety of different coupling devices that could be attached, either permanently or selectively, to lower opening 14 to carry gas to or from respiratory interface device 10. Thus, a variety of coupling devices (e.g., with or without swivels on one or both ends, and with or without an exhalation system formed integral to the device) may be used.

In an exemplary embodiment, shown in FIGS. 3-5, faceplate 12 is generally convex or bowl-shaped. This shape defines an interior space that accommodates a user's nose and other features when respiratory interface device 10A is in use. Faceplate 12 includes a peripheral end 18 that extends about faceplate 12. In this exemplary embodiment, faceplate peripheral end 18 extends generally towards the user's face when respiratory interface device 10 is in use. Faceplate peripheral end 18 includes an outer side 20, an inner side 22 (relative to the interior space), and a face side 24. That is, as used herein and with reference to faceplate 12, “outer” or “outwardly” means away from the interior space defined by bowl-shaped faceplate 12, and, “inner” or “inwardly” means toward the interior space defined by bowl-shaped faceplate 12. As is known, the faceplate 12 can be custom made to generally correspond to the user's respiratory interface device contour, 1A (in this embodiment). That is, the user's face is measured, e.g. scanned, and relevant dimensions are recorded. The general shape of faceplate 12, faceplate peripheral end 18, and faceplate peripheral end face side 24 generally correspond to the user's respiratory interface device contour 1A (in this embodiment).

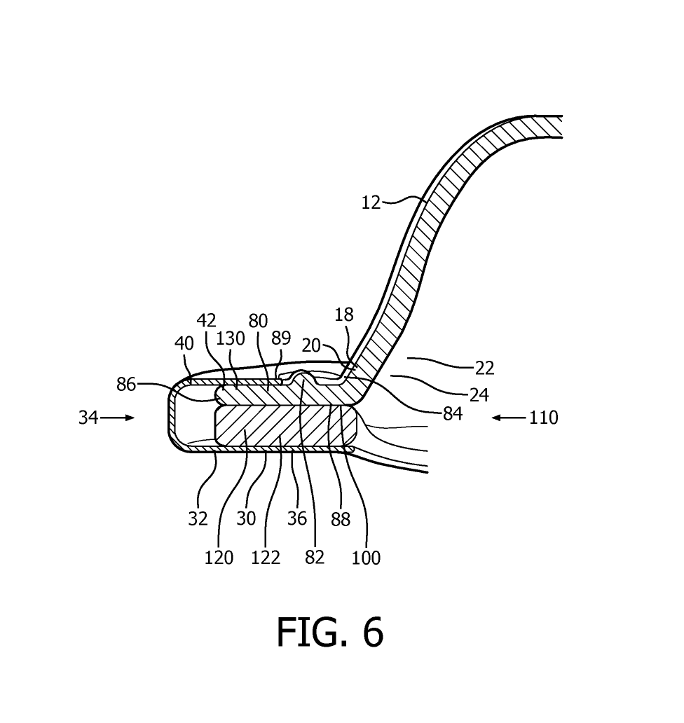

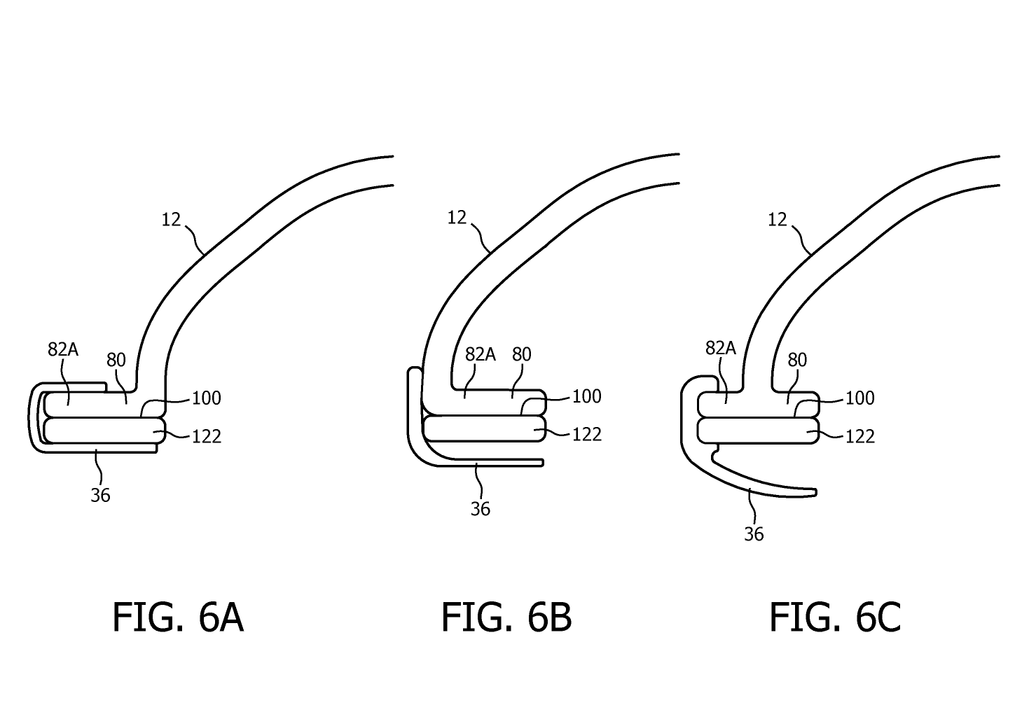

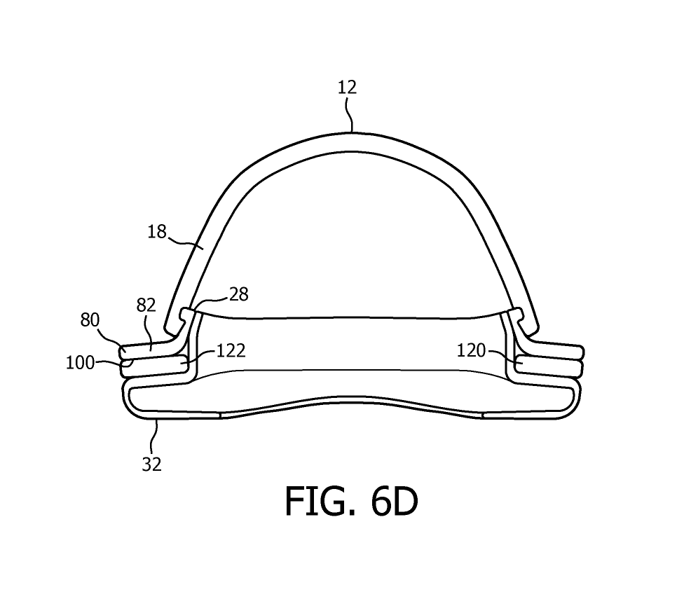

In an exemplary embodiment, faceplate peripheral end 18 is unitary with respiratory interface device cushion support assembly 80. That is, in an exemplary embodiment, faceplate 12 and respiratory interface device cushion support assembly 80 are made as a unitary body. In an alternative embodiment, cushion support assembly body 82 is coupled to faceplate peripheral end 18 and extends toward one of, or both, faceplate peripheral end outer side 20 or faceplate peripheral end inner side 22. Thus, faceplate peripheral end 18 includes a coupling component 28 (FIG. 6D) structured to be coupled, directly coupled, or fixed to respiratory interface device cushion support assembly 80.

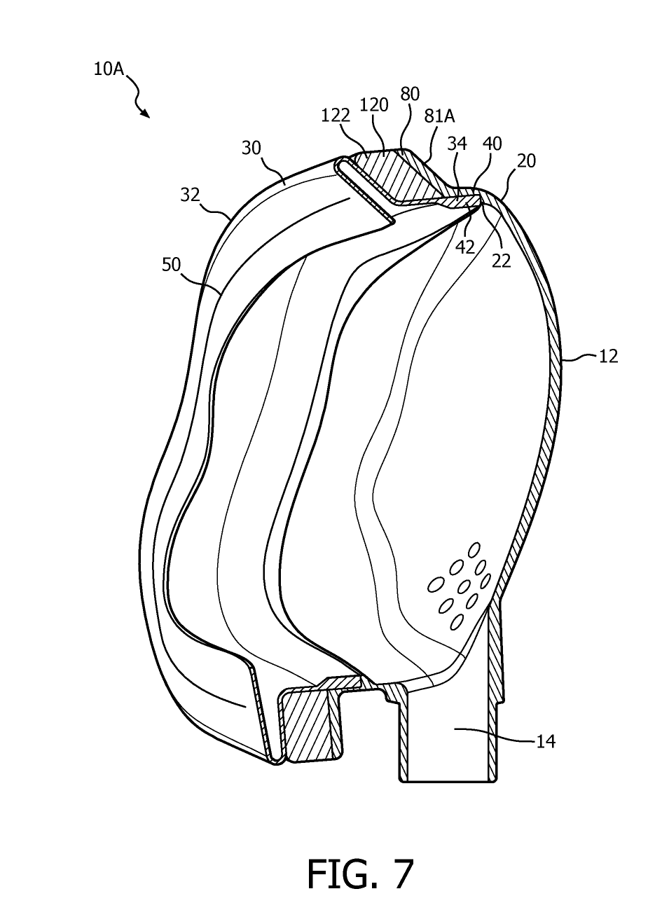

Respiratory interface device cushion 30 (hereinafter “cushion” 30) includes a body 32. Cushion body 32 can be constructed of a wide variety of resilient materials known in the art and can include, but is not limited to, a thermoplastic or thermoelastic material, including but not limited to an elastomer such as plastic, rubber, silicone, vinyl, foam, or any combination thereof. Cushion body 32 includes a coupling component 34 and an engagement portion 36. In the exemplary embodiment shown in FIG. 6, cushion body coupling component 34 is a groove 40 within cushion body 32 that is structured to be coupled to a tongue 130, described below. In an alternate embodiment, cushion body 32 has a generally C-shaped cross-section wherein cushion body 32 cross-sectional shape defines groove 40. In another alternate embodiment, shown in FIG. 7, cushion body coupling component 34 is a tongue 42 structured to be disposed in a groove 40, described below.

Cushion engagement portion 36 is structured to engage a user's face. Cushion engagement portion 36 is shaped to generally follow user's respiratory interface device contour, 1A (as well as 1B, 1C, 1D for other embodiments). That is, cushion engagement portion 36 includes a longitudinal axis 50 that generally conforms to the axial aspect of user's respiratory interface device contour, 1A (as well as 1B, 1C, 1D for other embodiments). That is, cushion body engagement portion longitudinal axis 50 generally matches the user's respiratory interface device contour, 1A (as well as 1B, 1C, 1D for other embodiments). Cushion engagement portion 36 is, in an unbiased state and when viewed along cushion body engagement portion longitudinal axis 50 generally planar.

Cushion support assembly 80 includes a body 82. Cushion support assembly body 82 is, in an exemplary embodiment, custom made for each user. That is, as noted above, a user's face may be measured, e.g. scanned, and relevant dimensions recorded. Cushion support assembly body 82 is then made so as to generally correspond to the size, shape, and contour of the user's face. In an exemplary embodiment, cushion support assembly body 82 is then made so as to generally correspond to the user's face at the user's respiratory interface device contour, 1A (in this embodiment).

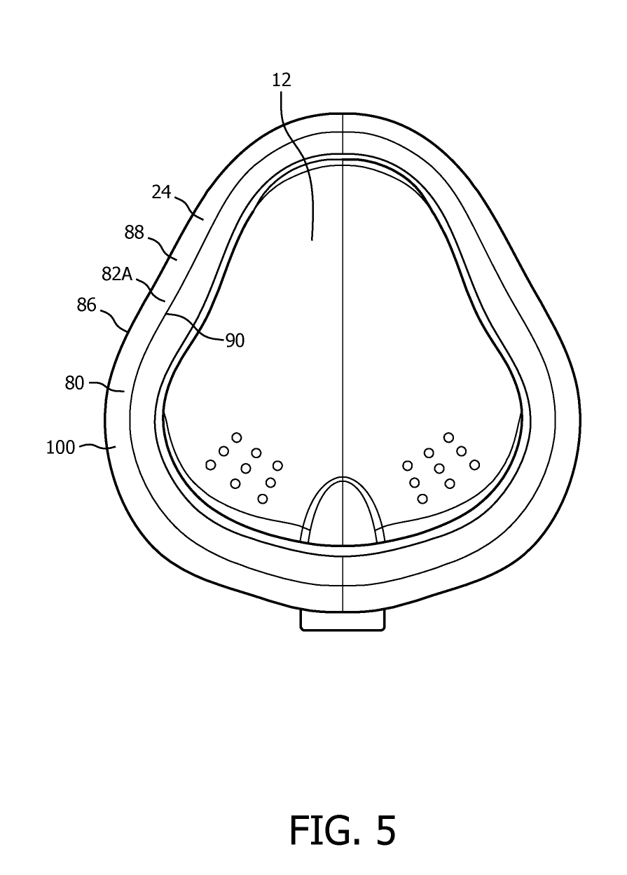

In an exemplary embodiment, as shown in FIGS. 5-7, cushion support assembly body 82 is a ribbon-like body 82A including an inner side 84, an outer side 86, a wide facial side 88, a wide exposed side 89 opposite facial side 88, and a longitudinal axis 90. Cushion support assembly ribbon-like body 82A is shaped to generally correspond to user's respiratory interface device contour axial aspect 2. That is, in this embodiment, cushion support assembly ribbon-like body 82A is shaped to extend about the user's nose and mouth. Stated alternately, cushion support assembly ribbon-like body longitudinal axis 90 generally corresponds to the user's respiratory interface device contour 1A (in this embodiment). In an exemplary embodiment, cushion support assembly ribbon-like body 82A is generally rectangular, and planar, when viewed along cushion support assembly ribbon-like body longitudinal axis 90 (FIG. 5). That is, in an exemplary embodiment, a cross-section of cushion support assembly ribbon-like body 82A is generally rectangular, and planar, when viewed along cushion support assembly ribbon-like body longitudinal axis 90.

Further, cushion support assembly ribbon-like body wide facial side 88 defines a support surface 100. Cushion support assembly ribbon-like body wide facial side support surface 100 (hereinafter “support surface” 100) is structured to support cushion engagement portion 36. In an exemplary embodiment, support surface 100 is structured to support cushion engagement portion 36 in a generally planar configuration. That is, as used herein, “to support cushion engagement portion 36 in a generally planar configuration” means that cushion engagement portion 36, when viewed along the longitudinal axis 50, is maintained in a generally planar configuration. It is understood that support surface 100 and cushion engagement portion 36 are not planar over their entire surface. That is, as set forth below, support surface 100 and cushion engagement portion 36 generally correspond to the user's respiratory interface device contour, and, support surface 100 and cushion engagement portion 36 each extend generally parallel to the user's face at any given location.

That is, support surface 100 extends in a direction that is generally planar relative to the user's respiratory interface device contour radial aspect. That is, as used herein, to be “generally planar relative to the user's respiratory interface device contour radial aspect” means that the surface of cushion body engagement portion 36 at any specific location extends generally parallel to the surface of the user's face at that specific location. In an exemplary embodiment, a segment of support surface 100 defines a plane. That is, a segment of support surface 100, such as but not limited to a segment having a length of about 0.1 mm along cushion support assembly ribbon-like body longitudinal axis 90, is generally a planar surface in the plane defined by the axial aspect and the radial aspect of the respiratory interface device contour 1A (in this embodiment). In this configuration, support surface 100 provides a planar support for support cushion engagement portion 36. That is, in an exemplary embodiment, support cushion engagement portion 36 has a width corresponding to the width of support surface 100 so that, when support cushion engagement portion 36 is disposed over support surface 100, support surface 100 maintains support cushion body engagement portion 36 in a generally planar configuration. A cushion body engagement portion 36 in a generally planar configuration provides a more complete seal compared to a cushion having a generally arcuate cross-sectional shape.

In an exemplary embodiment, cushion support assembly ribbon-like body 82A is structured to be coupled, directly coupled, or fixed to faceplate peripheral end 18. That is, cushion support assembly ribbon-like body 82A includes a coupling component 110 structured to be coupled, directly coupled, or fixed to faceplate peripheral end 18. In an exemplary embodiment, cushion support assembly ribbon-like body 82A coupling component 110 (hereinafter “cushion support assembly faceplate coupling component” 110) is structured to be coupled, directly coupled, or fixed to faceplate peripheral end coupling component 28. In an exemplary embodiment, cushion support assembly ribbon-like body 82A extends generally inward from faceplate peripheral end 18, that is, into the cavity defined by convex or bowl-shaped faceplate 12. In another exemplary embodiment, cushion support assembly ribbon-like body 82A extends generally outward from faceplate peripheral end 18, that is, away the cavity defined by convex or bowl-shaped faceplate 12. In another exemplary embodiment, faceplate peripheral end 18 is coupled to the medial portion of cushion support assembly body exposed side 89. Faceplate peripheral end coupling component 28 and cushion support assembly faceplate coupling component 110 can be any type of coupling components.

In another alternate embodiment, cushion support assembly ribbon-like body 82A is unitary with faceplate 12. As with the embodiments above, a cushion support assembly ribbon-like body 82A unitary with faceplate 12 can be configured to extend generally outward from faceplate peripheral end 18, as shown in FIG. 6A, extend generally inward from faceplate peripheral end 18, as shown in FIG. 6B, or extend both generally inward and outward from faceplate peripheral end 18, as shown in FIG. 6C.

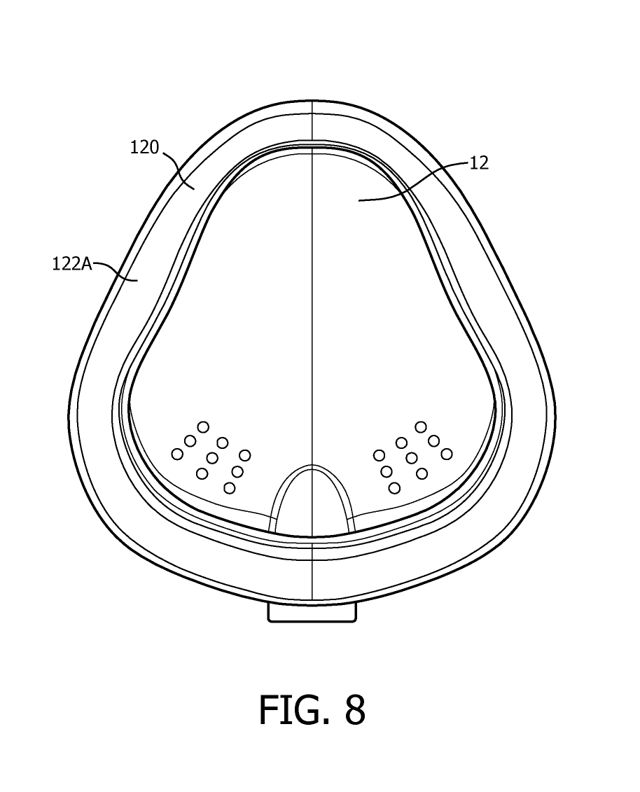

In an exemplary embodiment, as shown in FIGS. 7 and 8, cushion support assembly 80 includes a spring element 120. Cushion support assembly spring element 120 (hereinafter “spring element” 120) includes a resilient body 122. Spring element body 122, or alternately spring element 120, is structured to be, and is, disposed between support cushion body engagement portion 36 and support surface 100. Spring element body 122 is structured to conform to support surface 100; that is, in an exemplary embodiment, spring element body 122 that corresponds to support surface 100. Thus, spring element body 122 is shaped to generally correspond to user's respiratory interface device contour axial aspect 2. That is, in this embodiment, spring element body 122 is shaped to extend about the user's nose and mouth. Spring element body 122 is structured to be coupled, directly coupled, or fixed to support surface 100. In an exemplary embodiment, spring element body 122 can be constructed of a wide variety of resilient materials known in the art and can include, but is not limited to, a thermoplastic or thermoelastic material, including but not limited to an elastomer such as plastic, rubber, silicone, vinyl, foam, or any combination thereof.

In an exemplary embodiment, spring element body 122 is unitary with cushion body engagement portion 36. That is, in an exemplary embodiment, shown in FIG. 9, a lateral edge of cushion engagement portion 36, i.e. an edge extending generally parallel to cushion body engagement portion longitudinal axis 50, is unitary with spring element body 122. Thus, stated alternately, spring element body 122 is unitary with cushion body 32. Spring element body 122, in an exemplary embodiment, defines cushion body coupling component 34, as described above. For example, as shown in FIG. 10, spring element body 122 defines a groove 40. In this embodiment, support surface 100 which is generally planar acts as a tongue 130 sized to correspond to spring element body groove 40. Further, as shown in FIG. 11, support surface 100, in an exemplary embodiment, includes a number of longitudinal ridges 140 and channels 142. In this embodiment, spring element body groove 40, or stated more broadly cushion body 32, includes longitudinal ridges 144 and channels 146 that correspond to support surface longitudinal ridges 140 and channels 142.

When cushion body 32 is coupled to support surface 100, support surface longitudinal ridges 140 and channels 142 are disposed in cushion body longitudinal channels 146 and ridges 144, respectively. It is noted that support surface longitudinal ridges 140 and channels 142 are shallow relative to the thickness of cushion support assembly ribbon-like body 82A and, as used herein, support surface longitudinal ridges 140 and channels 142 do not change the nature of support surface 100 as “generally planar” as described above. As used herein, “shallow relative to the thickness of cushion support assembly ribbon-like body” means that support surface longitudinal ridges 140 and channels 142 have a depth/height less than 60% of the thickness of cushion support assembly ribbon-like body 82A.

As shown in FIGS. 9-13, there are several embodiments of spring element body 122 each with a different cross-sectional shape. In a first exemplary embodiment, shown in FIG. 9, spring element body 122 has a ribbon-like body 122A with a substantially rectangular cross-sectional shape. In this embodiment, spring element body 122A includes a first, wide cushion support assembly coupling surface 150 and an opposing wide cushion coupling surface 155. In this embodiment, spring element body cushion support assembly coupling surface 150 is coupled, directly coupled, or fixed to support surface 100. Further, spring element body cushion coupling surface 155 is coupled, directly coupled, or fixed to cushion body engagement portion 36.

In another exemplary embodiment, as shown in FIG. 14, spring element body 122″ has a substantially U-shaped cross-section. As used herein, a “U-shape” includes two elongated, generally parallel tines and a curved bight therebetween. Thus, the spring element body 122″ includes a first, wide cushion support assembly coupling tine 160 and an opposing wide cushion coupling tine 162. In this embodiment, spring element body cushion support assembly coupling tine 160 is coupled, directly coupled, or fixed to support surface 100. Further, spring element body cushion coupling tine 162 is coupled, directly coupled, or fixed to cushion body engagement portion 36.

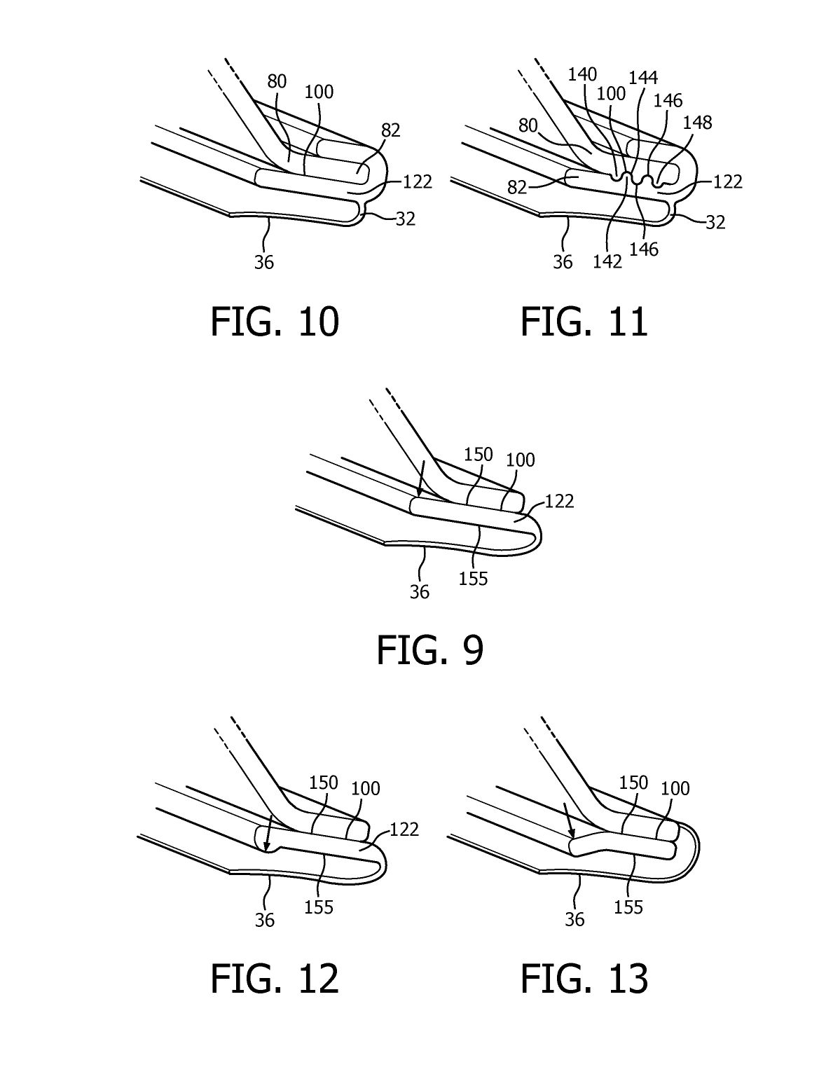

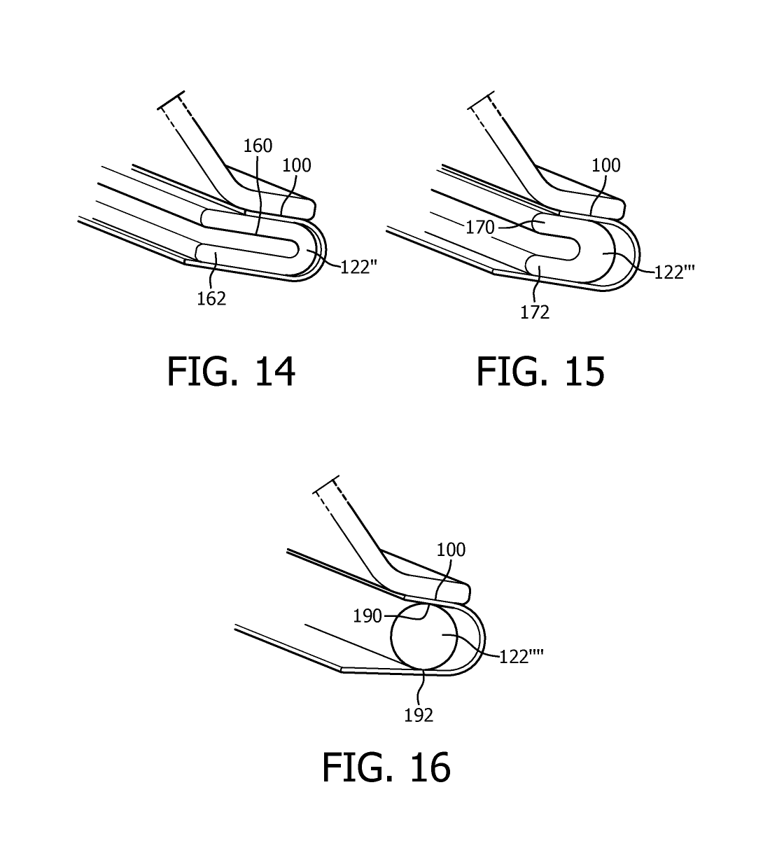

In another exemplary embodiment, shown in FIG. 15, spring element body 122′″ has a substantially C-shaped cross-section. As used herein, a “U-shape” includes two generally parallel tines and a curved bight therebetween. That is, unlike the “U-shape” described above, the tines are not elongated relative to the bight. Thus, the spring element body 122′″ includes a first, wide cushion support assembly coupling tine 170 and an opposing wide cushion coupling tine 172. In this embodiment, spring element body cushion support assembly coupling tine 170 is coupled, directly coupled, or fixed to support surface 100. Further, spring element body cushion coupling tine 172 is coupled, directly coupled, or fixed to cushion body engagement portion 36.

In another exemplary embodiment, shown in FIG. 16, spring element body 122″″ has a substantially circular cross-section when unbiased. In this embodiment, spring element body 122″″ is made from a highly deformable material, such as, but not limited to foam, TPE, gel, or silicone. Such materials have a durometer hardness of less than 10 A. As such, substantially circular cross-section spring element body 122″″ is structured to deform under sight bias so as to have a “pill-shaped” cross-sectional shape. As used herein, a “pill-shape” has two generally parallel surfaces maintained in spaced relationship with opposing semi-circular surfaces. In this exemplary embodiment, the pill-shaped cross-section spring element body 122″″ includes a first, wide cushion support assembly coupling surface 190 and an opposing wide cushion coupling surface 192. In this embodiment, spring element body cushion support assembly coupling surface 190 is coupled, directly coupled, or fixed to support surface 100. Further, spring element body cushion coupling surface 192 is coupled, directly coupled, or fixed to cushion body engagement portion 36.

In another embodiment, shown in FIGS. 17-18, respiratory interface device 10 is a cup-like nasal respiratory interface device 10B that is structured to be disposed over a user's nose. It is understood that reference numbers identify similar elements as discussed above and that for the embodiment shown in FIGS. 17-18 the reference numbers are followed by the letter “B.” Thus, for example, a “cushion support assembly ribbon-like body wide facial side” for this embodiment is identified by reference number 88B. In this embodiment, nasal respiratory interface device 10B is similar to the embodiment described above with the exception being that general shape of faceplate 12, faceplate peripheral end 18, and faceplate peripheral end face side 24 generally correspond to the user's respiratory interface device contour 1B (in this embodiment). Thus, cushion support assembly 80B also generally corresponds to the user's face at the user's respiratory interface device contour, 1B (in this embodiment).

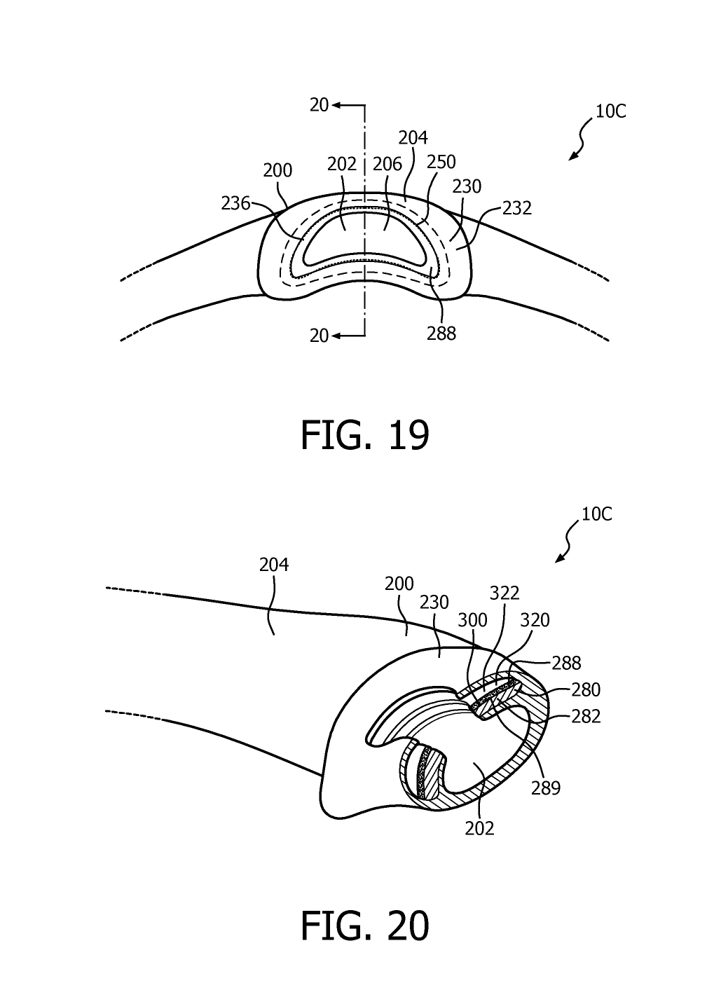

In another embodiment, shown in FIGS. 19-20, respiratory interface device 10 is a nasal respiratory interface device 10C in a “cradle” configuration. In this configuration, nasal respiratory interface device 10C is structured to be generally disposed below the patient's nose. Nasal respiratory interface device 10C includes an elongate hollow body 200 defining a cavity 202. Nasal respiratory interface device body 200 includes an upper side 204. Nasal respiratory interface device body upper side 204 defines an opening 206 into the nasal respiratory interface device cavity 202. In this embodiment, cushion 230 is disposed about, i.e. extending around, nasal respiratory interface device body opening 206. As before, cushion 230 includes a body 232 and an engagement portion 236.

In this embodiment, cushion body engagement portion 236 is structured to engage a user's face. Cushion engagement portion 236 is shaped to generally follow user's respiratory interface device contour 1C in this embodiment. That is, cushion engagement portion 236 includes a longitudinal axis 250 that generally conforms to user's respiratory interface device contour, 1C. Cushion engagement portion 236 is, in an unbiased state and when viewed along cushion engagement portion longitudinal axis 250 generally planar.

In this embodiment, cushion support assembly 280 includes a body 282. Cushion support assembly body 282 is, in an exemplary embodiment, custom made for each user. That is, as noted above, a user's face may be measured, e.g. scanned, and relevant dimensions recorded. Cushion support assembly body 282 is then made so as to generally correspond to the size, shape, and contour of the user's face. In an exemplary embodiment, cushion support assembly body 282 is then made so as to generally correspond to the user's face at the user's respiratory interface device contour, 1C (in this embodiment).

In an exemplary embodiment, cushion support assembly body 282 is a ribbon-like body 282 including a wide facial side 288, a wide interior side 289 opposite facial side 288, and a longitudinal axis. Cushion support assembly ribbon-like body 282C is shaped to generally correspond to user's respiratory interface device contour axial aspect 2. That is, in this embodiment, cushion support assembly ribbon-like body 282C is shaped to extend about the lower side of user's nose and about (around) the nostrils. Stated alternately, cushion support assembly ribbon-like body longitudinal axis 290 generally corresponds to the user's respiratory interface device contour 1C (in this embodiment). In an exemplary embodiment, cushion support assembly ribbon-like body 282 is generally rectangular, and planar, when viewed along cushion support assembly ribbon-like body longitudinal axis 290. That is, in an exemplary embodiment, a cross-section of cushion support assembly ribbon-like body 282 is generally rectangular, and planar, when viewed along cushion support assembly ribbon-like body longitudinal axis 90.

Further, cushion support assembly ribbon-like body wide facial side 288 defines a support surface 300. Cushion support assembly ribbon-like body wide facial side support surface 300 (hereinafter “support surface” 300) is structured to support cushion engagement portion 236. In an exemplary embodiment, support surface 300 is structured to support cushion engagement portion 236 in a generally planar configuration. That is, as used herein, “to support cushion engagement portion 236 in a generally planar configuration” means that cushion engagement portion 236, when viewed along the longitudinal axis 250, is maintained in a generally planar configuration. It is understood that support surface 300 and cushion engagement portion 236 are not planar over their entire surface. That is, support surface 300 and cushion engagement portion 236 generally correspond to the user's respiratory interface device contour 1C, and, support surface 300 and cushion engagement portion 236 each extend generally parallel to the user's face at any given location. That is, support surface 300 extends in a direction that is generally planar relative to the user's respiratory interface device contour radial aspect.

In an exemplary embodiment, cushion support assembly ribbon-like body 282C is structured to be coupled, directly coupled, or fixed to nasal respiratory interface device body 200.

As before, this exemplary embodiment may include a spring element 320. Cushion support assembly spring element 320 (hereinafter “spring element” 320) includes a resilient body 322. Spring element body 322, or alternately spring element 320, is structured to be, and is, disposed between support cushion engagement portion 236 and support surface 300. Spring element body 322 is structured to conform to support surface 300. That is, in an exemplary embodiment, spring element body 322 corresponds to support surface 300. Thus, spring element body 322 is shaped to generally correspond to user's respiratory interface device contour axial aspect 2. That is, in this embodiment, spring element body 322 is shaped to extend about the user's nostrils. Spring element body 322 is structured to be coupled, directly coupled, or fixed to support surface 300. In an exemplary embodiment, spring element body 322 can be constructed of a wide variety of resilient materials known in the art and can include, but is not limited to, a thermoplastic or thermoelastic material, including but not limited to an elastomer such as plastic, rubber, silicone, vinyl, foam, or any combination thereof.

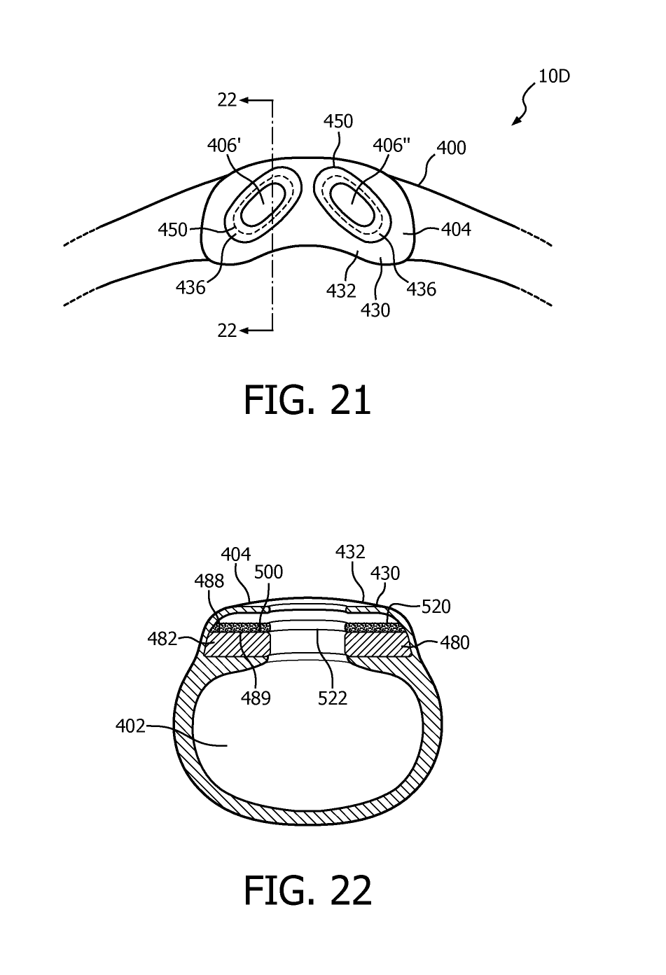

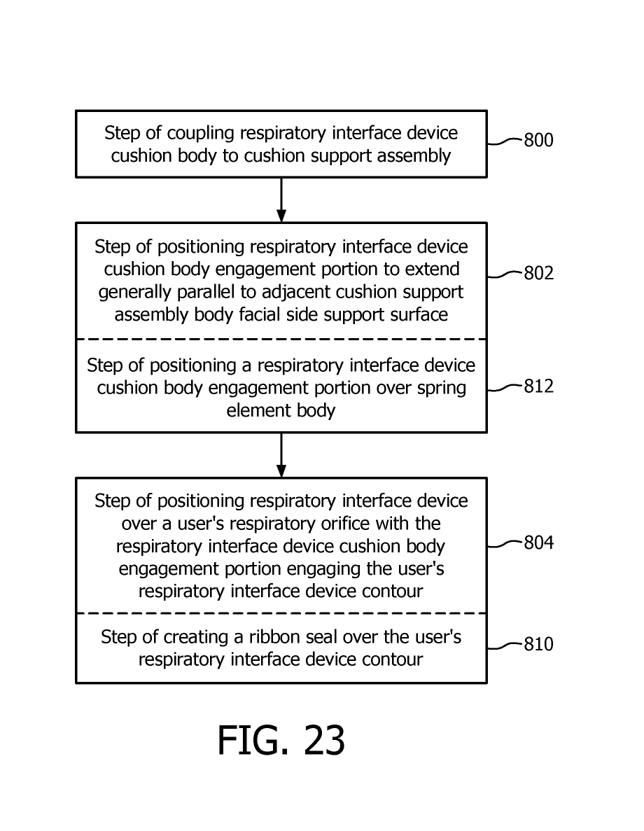

In another embodiment, shown in FIGS. 21-22, respiratory interface device 10 is a nasal respiratory interface device 10D in a sub-nasal surface sealing pillow configuration. As used herein, “sub-nasal surface sealing pillow” includes two pillows shaped to conform to the outer nasal surface. That is, unlike respiratory interface device pillows that extend into the nose, the “sub-nasal surface sealing pillows” are generally flat, but rise slightly above a generally planar surface.

In this configuration, nasal respiratory interface device 10D is structured to be generally disposed below the patient's nose. Nasal respiratory interface device 10D includes an elongate hollow body 400 defining a cavity 402. Nasal respiratory interface device body 400 includes an upper side 404. Nasal respiratory interface device body upper side 404 includes a surface defining two nostril openings 406′, 406″. In this embodiment, cushion 430 is disposed about, i.e. extending around, each nasal respiratory interface device body nostril opening 406′, 406″. As before, cushion 430 includes a body 432 and an engagement portion 436.

In this embodiment, cushion body engagement portion 436 is structured to engage a user's face. Cushion engagement portion 436 is shaped to generally follow user's respiratory interface device contour 1D in this embodiment. That is, cushion engagement portion 436 includes a longitudinal axis 450 that generally conforms to user's respiratory interface device contour, 1D. That is, for this embodiment, and for the purpose of using consistent terminology, as used herein, the “longitudinal axis” is a line forming two loops disposed about, i.e. extending around, a user's nostril openings; that is, the surface located on the outer portion and on the lower side of a user's nose. Cushion engagement portion 436 is, in an unbiased state and when viewed along cushion engagement portion longitudinal axis 450 generally planar.

In this embodiment, cushion support assembly 480 includes a body 482. Cushion support assembly body 482 is, in an exemplary embodiment, custom made for each user. That is, as noted above, a user's face may be measured, e.g. scanned, and a relevant dimensions recorded. Cushion support assembly body 482 is then made so as to generally correspond to the size, shape, and contour of the user's face. In an exemplary embodiment, cushion support assembly body 482 is then made so as to generally correspond to the user's face at the user's respiratory interface device contour, 1D (in this embodiment).

In an exemplary embodiment, cushion support assembly body 482 is a ribbon-like body 482 including a wide facial side 488, a wide interior side 489 opposite facial side 488, and a longitudinal axis. Again, as used herein, the “longitudinal axis” is a line corresponding to two loops disposed about, i.e. extending around, a user's nostril openings. Cushion support assembly ribbon-like body 482 is shaped to generally correspond to user's respiratory interface device contour axial aspect 2. That is, in this embodiment, cushion support assembly ribbon-like body 482 is shaped to extend about the lower side of user's nose and about (around) the nostrils. Stated alternately, cushion support assembly ribbon-like body longitudinal axis 290 generally corresponds to the user's respiratory interface device contour 1C (in this embodiment). In an exemplary embodiment, cushion support assembly ribbon-like body 482 is generally rectangular, and planar, when viewed along cushion support assembly ribbon-like body longitudinal axis. That is, in an exemplary embodiment, a cross-section of cushion support assembly ribbon-like body 482 is generally rectangular, and planar, when viewed along cushion support assembly ribbon-like body longitudinal axis 490.

Further, cushion support assembly ribbon-like body wide facial side 488 defines a support surface 500. Cushion support assembly ribbon-like body wide facial side support surface 500 (hereinafter “support surface” 500) is structured to support cushion engagement portion 436. In an exemplary embodiment, support surface 300 is structured to support cushion engagement portion 436 in a generally planar configuration. That is, as used herein, “to support cushion engagement portion 436 in a generally planar configuration” means that cushion body engagement portion 436, when viewed along the longitudinal axis 450, is maintained in a generally planar configuration. It is understood that support surface 500 and cushion engagement portion 436 are not planar over their entire surface. That is, support surface 300 and cushion body engagement portion 436 generally correspond to the user's respiratory interface device contour 1D, and, support surface 500 and cushion engagement portion 436 each extend generally parallel to the user's face at any given location. That is, support surface 500 extends in a direction that is generally planar relative to the user's respiratory interface device contour radial aspect.

In an exemplary embodiment, cushion support assembly ribbon-like body 482D is structured to be coupled, directly coupled, or fixed to nasal respiratory interface device body 500.

As before, this exemplary embodiment may include a spring element 520. Cushion support assembly spring element 520 (hereinafter “spring element” 520) includes a resilient body 522. Spring element body 522, or alternately spring element 520, is structured to be, and is, disposed between support cushion engagement portion 436 and support surface 500. Spring element body 522 is structured to conform to support surface 500. That is, in an exemplary embodiment, spring element body 522 corresponds to support surface 500. Thus, spring element body 522 is shaped to generally correspond to user's respiratory interface device contour axial aspect 2. That is, in this embodiment, spring element body 522 is shaped to extend about the user's nostrils. Spring element body 522 is structured to be coupled, directly coupled, or fixed to support surface 500. In an exemplary embodiment, spring element body 522 can be constructed of a wide variety of resilient materials known in the art and can include, but is not limited to, a thermoplastic or thermoelastic material, including but not limited to an elastomer such as plastic, rubber, silicone, vinyl, foam, or any combination thereof.

Accordingly, a method of using respiratory interface device 10, i.e. any of the embodiments described above, includes the following: coupling 800 respiratory interface device cushion body 32, 232, 432 to cushion support assembly 80, 280, 480, positioning 802 respiratory interface device cushion body engagement portion 36, 236, 426 to extend generally parallel to adjacent cushion support assembly body facial side support surface 100, 300, 500, positioning 804 respiratory interface device 10, 10A, 10B, 10C, 10D over a user's respiratory orifice with the respiratory interface device cushion body engagement portion 36, 236, 426 engaging the user's respiratory interface device contour 1A, 1B, 1C, 1D.

Further, when positioning 804 respiratory interface device 10, 10A, 10B, 10C, 10D over a user's respiratory orifice with the respiratory interface device cushion body engagement portion 36, 236, 426 engaging the user's respiratory interface device contour 1A, 1B, 1C, 1D, the method includes, creating 810 a ribbon seal over the user's respiratory interface device contour. Further, when positioning 802 respiratory interface device cushion body engagement portion 36, 236, 426 to extend generally parallel to adjacent cushion support assembly body facial side support surface 100, 300, 500, the method includes positioning 812 a respiratory interface device cushion body engagement portion 36, 236, 426 over a spring element body 122, 322, 522.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A cushion support assembly for a respiratory interface device cushion, the respiratory interface device cushion including a resilient body, the respiratory interface device cushion body including an engagement portion structured to engage a user's face, the user's face including a respiratory interface device contour, the user's respiratory interface device contour is a shaped loop including an axial aspect and a radial aspect, the respiratory interface device including a faceplate with a peripheral contour, the respiratory interface device cushion including a resilient body, the cushion support assembly comprising:
a cushion support assembly ribbon-like body including an inner side, an outer side and a wide facial side, wherein the cushion support assembly body facial side includes a support surface, the support surface structured to support the respiratory interface device cushion engagement portion in a generally planar configuration, and wherein the cushion support assembly body facial side support surface is generally planar relative to the user's respiratory interface device contour radial aspect.

2. The cushion support assembly of claim 1, wherein the cushion support assembly body is shaped to generally correspond to the user's respiratory interface device contour axial aspect.

3. The cushion support assembly of claim 1, wherein said cushion support assembly further comprises:
a spring element including a resilient body;
the spring element body structured to correspond to the cushion support assembly body facial side support surface; and
the spring element body coupled to the cushion support assembly body facial side support surface.

4. The cushion support assembly of claim 3, wherein the spring element body has a cross-sectional shape selected from the group including substantially rectangular, substantially U-shaped, and substantially C-shaped.

5. The cushion support assembly of claim 1, wherein the faceplate includes a peripheral end, and wherein the cushion support assembly body is structured to be coupled to the faceplate peripheral end.

6. A respiratory interface device structured to engage a user's face, the user's face including a respiratory interface device contour, the user's respiratory interface device contour is a shaped loop including an axial aspect and a radial aspect, comprising:
a faceplate including a body with a with a peripheral contour;
a respiratory interface device cushion including a resilient body, the respiratory interface device cushion body including an engagement portion structured to engage a user's face;
a cushion support assembly including a cushion support assembly ribbon-like body, wherein the cushion support assembly ribbon-like body includes an inner side and a facial side, wherein the cushion support assembly body facial side includes a support surface, wherein the support surface is structured to support the respiratory interface device cushion engagement portion, wherein the cushion support assembly body facial side support surface is generally planar relative to the user's respiratory interface device contour radial aspect, wherein the respiratory interface device cushion body is coupled to the cushion support assembly body, and wherein the respiratory interface device cushion body engagement portion extends substantially parallel to the cushion support assembly body facial side support surface.

7. The respiratory interface device of claim 6, wherein the cushion support assembly body is shaped to generally conform to the user's respiratory interface device contour axial aspect.

8. The respiratory interface device of claim 6, wherein:
the cushion support assembly includes a spring element;
the cushion support assembly spring element including a resilient body;
the spring element body structured to conform to the cushion support assembly body facial side support surface; and
the spring element body coupled to the cushion support assembly body facial side support surface.

9. The respiratory interface device of claim 8, wherein the spring element body has a cross-sectional shape selected from the group including substantially rectangular, substantially U-shaped, and substantially C-shaped.

10. The respiratory interface device of claim 6, wherein:
the faceplate includes a peripheral end; and
the cushion support assembly body is structured to be coupled to the faceplate peripheral end.

11. The respiratory interface device of claim 6, wherein the cushion support assembly body extends generally outwardly from the faceplate peripheral end.

12. The respiratory interface device of claim 6, wherein the cushion support assembly body extends generally inwardly from the faceplate peripheral end.

13. The respiratory interface device of claim 6, wherein:
the cushion support assembly body includes a cushion body coupling component; and
the respiratory interface device cushion body includes a coupling component.

14. The respiratory interface device of claim 13, wherein:
the cushion support assembly body cushion body coupling component is a portion of the cushion support assembly body structured to act as a tongue;
the respiratory interface device cushion body coupling component is a groove in the respiratory interface device cushion body;
the cushion support assembly body tongue corresponds to the respiratory interface device cushion body groove; and
the cushion support assembly body tongue is disposed in the respiratory interface device cushion body groove.

15. A method of using a respiratory interface device structured to engage a user's face, the user's face including a respiratory interface device contour, wherein the user's respiratory interface device contour is a shaped loop including an axial aspect and a radial aspect, the respiratory interface device including a faceplate, a cushion, and a cushion support assembly, the faceplate including a body with a with a peripheral contour, the respiratory interface device cushion including a resilient body, the respiratory interface device cushion body including an engagement portion structured to engage a user's face, the cushion support assembly including a cushion support assembly ribbon-like body, the cushion support assembly ribbon-like body including an inner side and a facial side, the cushion support assembly body facial side including a support surface, the support surface structured to support the respiratory interface device cushion engagement portion, wherein the cushion support assembly body facial side support surface is generally planar relative to the user's respiratory interface device contour radial aspect, the method including:
coupling the respiratory interface device cushion body to the cushion support assembly;
positioning the respiratory interface device cushion body engagement portion to extend generally parallel to the adjacent cushion support assembly body facial side support surface;
positioning the respiratory interface device over a user's respiratory orifice with the respiratory interface device cushion body engagement portion engaging the user's respiratory interface device contour.

16. The method of claim 15, wherein the step of positioning the respiratory interface device over a user's respiratory orifice with the respiratory interface device cushion body engagement portion engaging the user's respiratory interface device contour includes the step of creating a ribbon seal over the user's respiratory interface device contour.

17. The method of claim 15, wherein the cushion support assembly includes spring element, the cushion support assembly spring element including a resilient body, the spring element body structured to conform to the cushion support assembly body facial side support surface, and the spring element body coupled to the cushion support assembly body facial side support surface, and wherein the step of positioning the respiratory interface device cushion body engagement portion to extend generally parallel to the adjacent cushion support assembly body facial side support surface includes the step of positioning a respiratory interface device cushion body engagement portion over a spring element body.
